(19) United States
(12) Patent Application Publication
(10) Pub. No.: US 2024/0122507 A1
(43) Pub. Date: Apr. 18, 2024

(54) BIOSENSOR PLACEMENT

(71) Applicant: BIOINTELLISENSE, INC., Golden, CO (US)

(72) Inventors: Henry Do, Los Angeles, CA (US); Harley Gordon White, Fort Collins, CO (US); Zongde Qiu, Cupertino, CA (US); David Jonq Wang, Palo Alto, CA (US); Alexander Katsis, San Mateo, CA (US); Thomas Dietiker, Palos Verdes Estates, CA (US)

(21) Appl. No.: 18/047,211

(22) Filed: Oct. 17, 2022

(51) Int. Cl. A61B 5/1455; A61B 5/00

(52) U.S. Cl. CPC A61B 5/14551; A61B 5/6826; A61B 5/02416

(57) ABSTRACT

An example method includes generating a signal correlated with blood perfusion or SpO₂ of a subject at a location and orientation on a body of the subject; determining a signal quality and/or signal strength of the signal; and in dependence on the signal quality and/or signal strength, either indicating that at least one of the location or the orientation is acceptable for placement of a biosensor on the body of the subject or prompting the subject or other person to reposition the biosensor. The biosensor may be part of a chest sensor device that includes: an adhesive with a liner having pull tabs for easy removal when the chest sensor device is positioned at a desired location; a finger grip to facilitate removal of the chest sensor device from the subject after a period of use; and/or wraparound fingers to secure the chest sensor device to the adhesive.

### CROSS-REFERENCE TO RELATED APPLICATION

This application is related to U.S. Provisional Patent Application No. 63/371,830 filed Aug. 18, 2022 and which is incorporated herein by reference in its entirety.

### FIELD

The embodiments discussed herein are related to a biosensor placement.

### BACKGROUND

Unless otherwise indicated herein, the materials described herein are not prior art to the claims in the present application and are not admitted to be prior art by inclusion in this section.

Biosensors commonly measure peripheral oxygen saturation (SpO₂) as well as other physiological parameters using optical emitters such as light emitting diodes (LEDs) of different peak wavelengths together with optical detectors such as photodiodes to measure the absorption of multiple wavelengths (e.g., red and infrared (IR)) of light at a location on a body. For example, oxygenated hemoglobin absorbs more IR light than red light and deoxygenated hemoglobin absorbs more red light than IR light, so SpO₂ of a subject's blood may be determined by measuring the absorbance of red and IR light and calculating SpO₂ based on the absorbance of the two wavelengths of light. Alternatively, at least some biosensors calculate SpO₂ based on reflectance based absorption of red light and IR light. Subjects seeking to benefit from use of biosensors often have difficulty in placing the biosensor at a location with adequate blood perfusion (e.g., over an intercostal space (ICS)). Measuring SpO₂ at locations on the body with little or no blood perfusion may result in inaccurate or absent readings.

The subject matter claimed herein is not limited to implementations that solve any disadvantages or that operate only in environments such as those described above. Rather, this background is only provided to illustrate one example technology area where some implementations described herein may be practiced.

### SUMMARY

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

In an example embodiment, a method to monitor placement of a biosensor includes generating, using the biosensor, a signal correlated with blood perfusion or peripheral oxygen saturation (SpO₂) of a subject at a location and orientation on a body of the subject. The method determining at least one of a signal quality or signal strength of the signal. The method includes, in dependence on at least one of the signal quality or the signal strength, either indicating that at least one of the location or orientation is acceptable for placement of the biosensor on the body of the subject or prompting the subject or other person to reposition the biosensor to a different location or orientation on the body of the subject.

In another example embodiment, a method includes positioning a chest sensor device including a biosensor at a location on a body of a subject. The chest sensor device has an orientation with respect to the body at the location. The method includes receiving output of the chest sensor device indicative of whether at least one of the location or orientation of the chest sensor device on the body of the subject is acceptable for placement of the chest sensor device. The method includes, in response to the output indicating that at least one of the location or orientation of the chest sensor device is acceptable, affixing the chest sensor device to the body of the subject at the location and orientation.

In another example embodiment, a method includes positioning a chest sensor device including a biosensor at a location on a body of a subject. The chest sensor device includes an adhesive with a liner having a pull tab that extends beyond a perimeter of the chest sensor device. The method includes affixing the chest sensor device to the body of the subject at the location, including using the pull tab to remove the liner while holding the chest sensor device in place at the location on the body of the subject. The adhesive couples the chest sensor device to the body at the location in the absence of the liner.

In another example embodiment, a chest sensor device includes a biosensor, a processor, and an output device. The biosensor is configured to generate a signal correlated with blood perfusion or SpO₂ of a subject at a location and orientation on a body of the subject. The processor is configured to determine a signal quality of the signal and whether the signal quality is acceptable. The output device is configured to output, in response to determining that the signal quality is acceptable, an indication that at least one of the location or the orientation is acceptable for placement of the biosensor on the body of the subject.

In another example embodiment, a chest sensor device includes a first adhesive surface, a second adhesive surface, and a liner. The first adhesive surface is configured to be adhered to skin of a subject. The second adhesive surface is opposite the first adhesive surface and is configured to be adhered to a biosensor. The liner is removably coupled to the first adhesive surface and includes at least a first pull tab configured to allow the subject to remove the liner without displacing the biosensor relative to the subject.

In another example embodiment, a chest sensor device includes a first adhesive surface, a second adhesive surface, and a finger grip. The first adhesive surface is configured to be adhered to skin of a subject and is made of a first material. The second adhesive surface is opposite the first adhesive surface and is configured to be adhered to a biosensor. The finger grip is made of a second material that is different than the first material. The finger grip is coupled to the first and second adhesive surfaces and lacks any adhesive.

In another example embodiment, a chest sensor device includes a housing, a biosensor, an adhesive patch, and a wraparound finger. The biosensor is at least partially disposed in the housing. The adhesive patch is coupled to the housing and includes first and second adhesive surfaces. The first adhesive surface is configured to be adhered to skin of a subject. The second adhesive surface is opposite the first adhesive surface and is adhered to the housing. The wraparound finger extends from the adhesive patch. The wraparound finger at least partially wraps around the housing from a bottom of the housing across a side of the housing to a top of the housing.

In another example embodiment, a method of placing a chest sensor device on a body of a subject includes removing a first liner from a first side of the adhesive. The adhesive includes a second side opposite the first side, the second side at least partially covered by a second liner. The method includes adhering the first side of the adhesive to the chest sensor device. The method includes placing the chest sensor device and adhesive near or in contact with a location on the body of the subject. The method includes, while holding the chest sensor device and adhesive in place in close proximity to the location on the body, pulling one or more liner tabs of the second liner to pull the second liner away from the second side of the adhesive. The method includes pressing the chest sensor device and adhesive onto the body at the location to obtain adhesive to skin adherence at the location.

device to align a biosensor superficial to ICS of a body of a subject includes a biosensor, a processor, and an output device. The biosensor is configured to generate a signal representing blood perfusion of the subject at a location on the body of the subject based on light detected by an optical detector of the biosensor. The processor is configured to determine Perfusion-Index (PI) at the location on the body of the subject based on the signal generated by the sensor. The output device is configured to output an indication of a value of the PI.

In another example embodiment, a chest sensor device includes a first adhesive surface, a second adhesive surface, and a liner. The first adhesive surface is configured to be adhered to skin of a subject. The second adhesive surface is configured to be adhered to a biosensor. The liner is removably coupled to the first adhesive surface and includes a pull tab. The pull tab is configured to allow the subject to remove the liner without displacing the biosensor relative to the subject.

In another example embodiment, a chest sensor device includes a first adhesive surface, a second adhesive surface, and a finger grip. The first adhesive surface is configured to be adhered to skin of a subject and is made of a first material. The second adhesive surface is opposite the first adhesive surface and is configured to be adhered to a biosensor. The finger grip is made of a second material that is different than the first material.

Additional features and advantages of these embodiments will be set forth in the description which follows, and in part will be obvious from the description, or may be learned by the practice of the embodiments. The features and advantages of these embodiments may be realized and obtained by means of the instruments and combinations particularly pointed out in the appended claims. These and other features of the present embodiments will become more fully apparent from the following description and appended claims or may be learned by the practice of the embodiments as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 illustrates an example operating environment for a chest sensor device;

FIG. 2 illustrates an example chest sensor device that may be implemented in the environment of FIG. 1;

FIG. 3A is a flowchart of an example method to monitor placement of a biosensor on a body of a subject;

FIG. 3B is a flowchart of another example method to monitor placement of a biosensor on a body of a subject;

FIG. 3C is a flowchart of another example method to monitor placement of a biosensor on a body of a subject;

FIG. 4A is a flowchart of an example method to place a chest sensor device on a subject;

FIG. 4B is a flowchart of an example method to remove an adhesive liner from chest sensor device without disrupting its placement;

FIG. 5 illustrates another example chest sensor device that may be implemented in the environment of FIG. 1;

FIGS. 6A and 6B illustrate a front view and a back view of another example chest sensor device that may be implemented in the environment of FIG. 1;

FIGS. 7A-7C illustrate another example chest sensor device that may be implemented in the environment of FIG. 1;

FIGS. 8A and 8B illustrate another example chest sensor device that may be implemented in the environment of FIG. 1;

FIG. 9 illustrates an example finger grip for a chest sensor device that may be implemented in the environment of FIG. 1; and

FIGS. 10A and 10B illustrate another example chest sensor device that may be implemented in the environment of FIG. 1;

FIG. 11 is a block diagram illustrating an example computing device,

all arranged in accordance with at least one embodiment described herein.

### DETAILED DESCRIPTION OF SOME EXAMPLE EMBODIMENTS

Many biosensors have an array of light emitting diodes (LEDs) used in measuring SpO₂. These biosensors are typically large and not immune to motion. Other biosensors may be smaller and/or less affected by motion but may be more sensitive to placement on a subject. For example, if a biosensor is placed at a location on the body of the subject with limited blood perfusion (such as directly over a rib), blood flow at the measurement site may be insufficient to generate accurate SpO₂ measurements.

To mitigate improper placement of such biosensors, some embodiments herein incorporate the biosensor and one or more output devices into a chest sensor device. The one or more output device may be configured to indicate to the subject the value of the Perfusion-Index (PI) at a location on the subject's body or to otherwise indicate whether the location is a good location for measurements. The indication from the one or more output devices may be configured to improve a likelihood of the subject aligning a biosensor superficial to the intercostal space (ICS) between two ribs for accurate SpO₂ measurements. For example, the one or more output devices may give a visual, auditory, and/or haptic indicator to the subject when the biosensor is at a location with a large PI (e.g., a PI greater than a threshold) and give a different visual, auditory, and/or haptic indictor when the biosensor is at a location with a small PI (e.g., a PI less than the threshold). The chest sensor device may include one or more biosensors positioned at a first surface to be proximate to the subject's skin when the chest sensor device is coupled to the subject and an opposing second surface. The second surface may include the one or more output devices to use as an aid in positioning and/or aligning the one or more biosensors between ribs to be directly over a corresponding ICS.

Reference will now be made to the drawings to describe various aspects of example embodiments of the invention. It is to be understood that the drawings are diagrammatic and schematic representations of such example embodiments, and are not limiting of the present invention, nor are they necessarily drawn to scale.

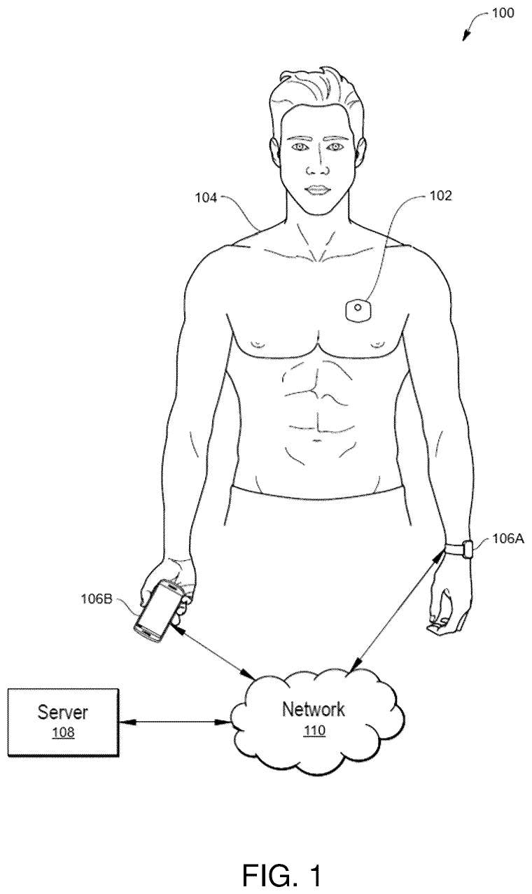

FIG. 1 illustrates an example operating environment 100 (hereinafter “environment 100”) for a chest sensor device 102, arranged in accordance with at least one embodiment described herein. The environment 100 includes a subject 104 and one or more personal electronic devices 106A, 106B (hereinafter collectively “personal electronic devices 106” or generically “personal electronic device 106”). The environment 100 may additionally include a server 108 and a network 110.

In general, the chest sensor device 102 may be attached to the subject 104, such as to the chest of the subject 104, to detect blood perfusion, oxygen saturation of hemoglobin, and/or other measurement parameters. In an example, the chest sensor device 102 is configured specifically to detect blood perfusion of the subject 104. In some embodiments, the quality of measurements generated by the chest sensor device 102 may depend on the position of the chest sensor device 102 relative to the ribs and/or ICSs of the rib cage of the subject 104.

The personal electronic devices 106 may each include a desktop computer, a laptop computer, a tablet computer, a smartphone, a wearable electronic device (e.g., smart watch, activity tracker, headphones, ear buds, etc.), or other personal electronic device. In the illustrated example, the personal electronic device 106A is a smart watch and the personal electronic device 106B is a smartphone. In some embodiments, the personal electronic devices 106 may collect measurement data from the chest sensor device 102 for use and/or analysis on the personal electronic devices 106.

Alternatively or additionally, the measurement data generated by the chest sensor device 102 and/or data derived therefrom may be uploaded, e.g., periodically, by the chest sensor device 102 to the server 108. In some embodiments, one or more of the personal electronic devices 106 or another device may act as a hub that collects measurement data or data derived therefrom from the chest sensor device 102 and/or other personal electronic devices 106 and uploads the measurement data or data derived therefrom to the server 108. For example, the hub may collect data over a local communication scheme (WI-FI, BLUETOOTH, near-field communications (NFC), etc.) and may transmit the data to the server 108. In some embodiments, the hub may act to collect the data and periodically provide the data to the server 108, such as once per week. An example hub and associated methods and devices are disclosed in U.S. Pat. No. 10,743,091, which is incorporated herein by reference.

The server 108 may include a collection of computing resources available in the cloud v and/or a discrete server computer. The server 108 may be configured to receive measurement data and/or data derived from measurement data from one or more of the personal electronic devices 106 and/or from the chest sensor device 102. Alternatively and/or additionally, the server 108 may be configured to receive from the chest sensor device 102 (e.g., directly or indirectly via a hub device) relatively small portions of the measurement data, or even larger portions or all of the measurement data. The server 108 may use and/or analyze the data, e.g., to detect and/or monitor blood perfusion of the subject 104 or other biological parameters (e.g., oxygen saturation, heart rate, arrhythmia, or the like). Alternatively and/or additionally, the server 108 may store the measurement data in an account of the subject 104 and make the measurement data or data derived therefrom available to the subject 104, a healthcare provider, or other individuals, e.g., as authorized by the subject 104 e.g., via an online portal.

The network 110 may include one or more wide area networks (WANs) and/or local area networks (LANs) that enable the personal electronic devices 106, the server 108, and/or the chest sensor device 102 to communicate with each other. In some embodiments, the network 110 includes the Internet, including a global internetwork formed by logical and physical connections between multiple WANs and/or LANs. Alternately or additionally, the network 110 may include one or more cellular radio frequency (RF) networks and/or one or more wired and/or wireless networks such as 801.xx networks, BLUETOOTH access points, wireless access points, IP-based networks, or other suitable networks. The network 110 may also include servers that enable one type of network to interface with another type of network.

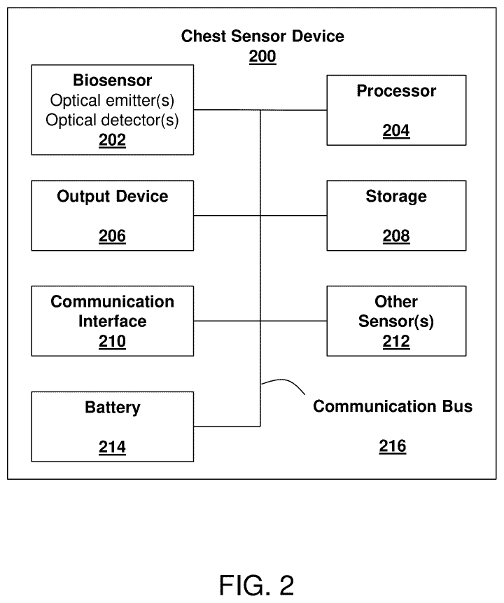

FIG. 2 illustrates an example chest sensor device 200 that may be implemented in the environment 100 of FIG. 1, arranged in accordance with at least one embodiment described herein. FIG. 2 includes a block diagram of the chest sensor device 200. The chest sensor device 200 may include, be included in, or correspond to the chest sensor device 102 of FIG. 1 and/or other chest sensor devices described herein.

In general, the chest sensor device 200 may include a biosensor 202, a processor 204, an output device 206, storage 208, a communication interface 210, other sensor(s) 212, a battery 214, and a communication bus 216. The biosensor 202 may include one or more optical emitters and one or more optical detectors. The optical emitters may be monochromatic light sources, non-monochromatic or broadband light sources, adjustable wavelength light sources, or the like. For example, the optical emitters may each include a single monochromatic light source such as a red or infrared (IR) light emitting diode (LED), multiple monochromatic light sources of different wavelengths such as a red LED, an IR LED, and/or a white LED (or other broad spectrum emitter), an adjustable wavelength light source such as a single LED adjustable between red and IR wavelengths, or any combination thereof. The optical detectors may each include a single optical detector, multiple optical detectors, and/or multiple optical detectors with different spectral response curves. Alternatively and/or additionally, each of the optical detectors may include a dual photodiode, a dual junction wavelength detector, an epitaxial spectral sensor, or other suitable optical detector. In some embodiments, each of the optical detectors includes a first detector and a second detector having different spectral response curves. Alternative and/or additional details regarding example optical sensors according to some embodiments herein are disclosed in U.S. Pat. No. 10,485,463 (hereinafter the '463 patent), which is incorporated herein by reference.

In general, embodiments disclosed in the '463 patent and which may be implemented herein may leverage a spectral sensor (e.g., one of the optical detector(s) of the biosensor 202) and a white LED (e.g., one of the optical emitter(s) of the biosensor 202) to obtain photoplethysmograph (PPG) waveforms (e.g., the “signals” generated herein) and from which SpO₂ measurements may be derived that are virtually free of motion interference. Such PPG waveforms may be processed instead of or in addition to one or more specific signals derived from red, IR, or other wavelengths. For example, embodiments in the '463 patent and/or herein may eliminate error when using a non-invasive optical sensing system such as the biosensor 202 by providing a first detector and a second detector in a non-invasive optical sensor (e.g., the biosensor 202), the first detector and the second detector having different respective spectral response curves. Light may be emitted from a non-monochromatic light source (e.g., a white LED or other broad spectrum emitter) in the non-invasive optical sensor periodically. Red light and infrared light may be emitted from one or more monochromatic light sources (e.g., an adjustable LED, a red LED, an IR LED, or the like). A first oxygen saturation value may be generated based on a ratio of the red light detected by the first and second detectors, and the infrared light detected by the first and second detectors. A second oxygen saturation value may be generated based on the non-monochromatic light detected by the first and second detectors. The first oxygen saturation value may be substituted with the second oxygen saturation value in response to a determination that a ratio of a first measured centroid wavelength of the red light by the first detector and a second measured centroid wavelength of the red light by the second detector is outside of a threshold. Additional example aspects of the foregoing that may be implemented herein are disclosed in the '463 patent.

With continued reference to FIG. 2, the processor 204 may include any device or component configured to monitor and/or control operation of the chest sensor device 200. In some embodiments, the processor 204 is electrically coupled to the biosensor 202 and is configured to control the biosensor 202 to generate a signal corresponding to or representing PI. In these and other embodiments, the processor 204 may retrieve instructions from the storage 208 and execute those instructions. As another example, the processor 204 may read the signals and/or measurement data generated by the biosensor 202 and/or other sensor(s) 212, process the signals and/or measurement data to generate other measurement data, and store any of the foregoing in the storage 208 or instruct the communication interface 210 to send any of the foregoing to another electronic device, such as the server 108 of FIG. 1. In some embodiments, the processor 204 may include an arithmetic logic unit, a microprocessor, a general-purpose controller, or some other processor or array of processors, to perform or control performance of operations as described herein. The processor 204 may be configured to process data signals and may include various computing architectures including a complex instruction set computer (CISC) architecture, a reduced instruction set computer (RISC) architecture, or an architecture implementing a combination of instruction sets. Although illustrated as a single processor 204, multiple processor devices may be included and other processors and physical configurations may be possible. The processor 204 may be configured to process any suitable number format including, but not limited to two's compliment numbers, integers, fixed binary point numbers, and/or floating point numbers, etc. all of which may be signed or unsigned. In some embodiments, the processor 204 may perform processing on the readings from the sensors prior to storing and/or communicating the readings. For example, raw analog data signals generated by the biosensor 202 or the other sensor(s) 212 may be down-sampled, may be converted to digital data signals, and/or may be processed in some other manner.

The storage 208 may include non-transitory computer-readable storage media or one or more non-transitory computer-readable storage mediums for carrying or having computer-executable instructions or data structures stored thereon. Such non-transitory computer-readable storage media may be any available non-transitory media that may be accessed by a general-purpose or special-purpose computer, such as the processor 204. By way of example such non-transitory computer-readable storage media may include Random Access Memory (RAM), Read-Only Memory (ROM), Electrically Erasable Programmable Read-Only Memory (EEPROM), flash memory devices (e.g., solid state memory devices), or any other non-transitory storage medium which may be used to carry or store desired program code in the form of computer-executable instructions or data structures and which may be accessed by a general-purpose or special-purpose computer. In some embodiments, the storage 208 may alternatively or additionally include volatile memory, such as a Dynamic Random Access Memory (DRAM) device, a Static Random Access Memory (SRAM) device, or the like. Combinations of the above may also be included within the scope of non-transitory computer-readable storage media. Computer-executable instructions may include, for example, instructions and data that when executed by the processor 204 cause the processor 204 to perform or control performance of a certain operation or group of operations. In some embodiments, the storage 208 may store the data signals, e.g., measurement data, generated by the biosensor 202, the other sensor(s) 212, and/or data derived therefrom.

The communication interface 210 may include any device or component that facilitates communication with a remote device, such as any of the personal electronic devices 106 of the subject 104, the server 108, or any other electronic device. For example, the communication interface 210 may include an RF antenna, an infrared (IR) receiver, a WI-FI chip, a BLUETOOTH chip, a cellular chip, a near-field communication (NFC) chip, or any other communication interface.

The battery 214 may include any device or component configured to provide power to the chest sensor device 200 and/or the components thereof. For example, the battery 214 may include a rechargeable battery, a disposable battery, etc. In some embodiments, the chest sensor device 200 may include circuitry, electrical wires, etc. to provide power from the battery 214 to the other components of the chest sensor device 200. In some embodiments, the battery 214 may include sufficient capacity such that the chest sensor device 200 may operate for days, weeks, or months without having the battery 214 changed or recharged. For example, the chest sensor device 200 may be configured to operate for at least two months without having the battery 214 charged or replaced.

The communication bus 216 may include any connections, lines, wires, or other components facilitating communication between the various components of the chest sensor device 200. The communication bus 216 may include one or more hardware components and may communicate using one or more protocols. Alternatively and/or additionally, the communication bus 216 may include wire connections between the components. In some embodiments, the chest sensor device 200 may operate in a similar or comparable manner to the embodiments described in U.S. application Ser. No. 17/485,315 filed on Sep. 24, 2021, U.S. application Ser. No. 17/349,166 filed on Jun. 16, 2021, and/or U.S. Pat. No. 11,172,909 issued Nov. 16, 2021, each of which is hereby incorporated by reference.

The one or more other sensor(s) 212 may include an electrocardiogram (ECG) sensor, a temperature sensor, a respiratory sensor, an accelerometer, a microphone, a gyrometer sensor, a blood pressure sensor, an optical spectrometer sensor, an electro-chemical sensor, an oxygen saturation sensor, an electrodermal activity (EDA) sensor, a volatile organic compound (VOC) sensor, a spectrometer, or any combination thereof. An ECG sensor may be configured to detect electrical activity of a subject's heart. A temperature sensor may be configured to detect temperatures associated with the subject, such as skin temperature and/or core body temperature. A respiratory sensor may be configured to detect respiration of the subject. An accelerometer may be configured to detect movement of at least a portion of the subject. A microphone may be configured to detect sound. A gyrometer sensor may be used to measure angular velocity of at least a portion of the subject, such as the chest of subject. An oxygen saturation sensor may be used to record blood oxygenation of the subject. An EDA sensor may be used to measure EDA of the skin of the subject. A VOC detector may be used to detect various organic molecules that may be coming off of the subject or that may be in the subject's sweat. An optical sensor may be used to monitor or detect changes in color, such as changes in skin coloration of the subject. A spectrometer may measure electromagnetic (EM) radiation and may be configured to detect variations in reflected EM radiation. For example, such a sensor may detect changes in color in a molecule exposed to multi-spectral light (e.g., white light), and/or may detect other changes in reflected EM radiation outside of the visible spectrum (e.g., interaction with ultra-violet rays, etc.).

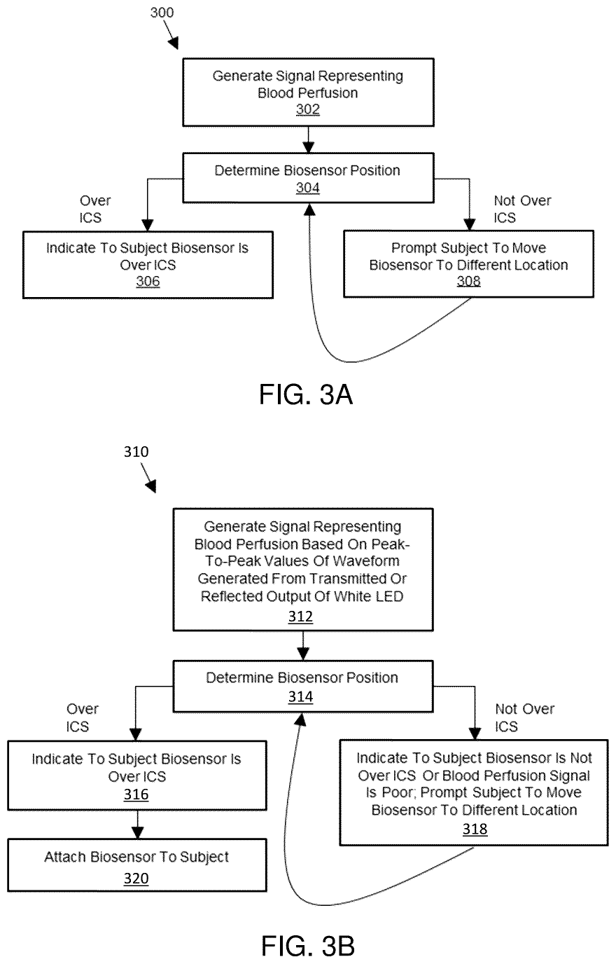

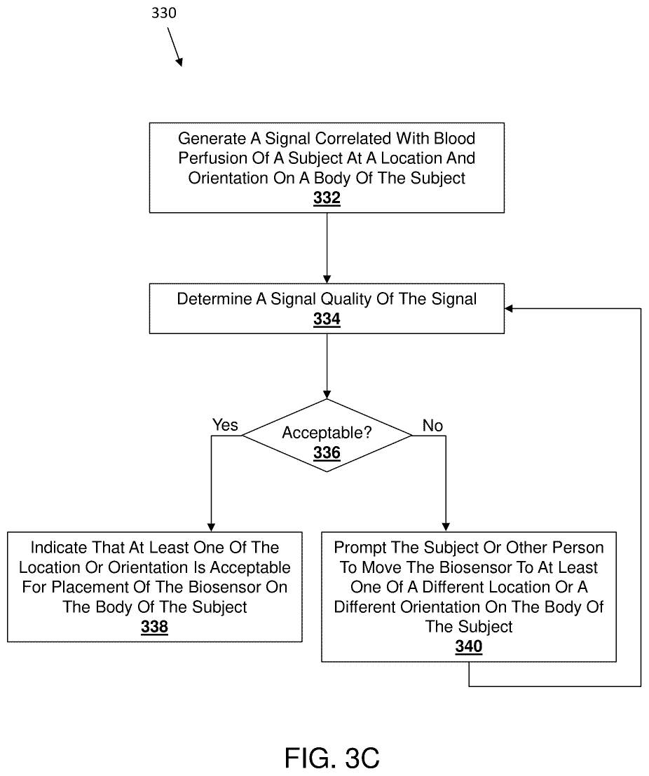

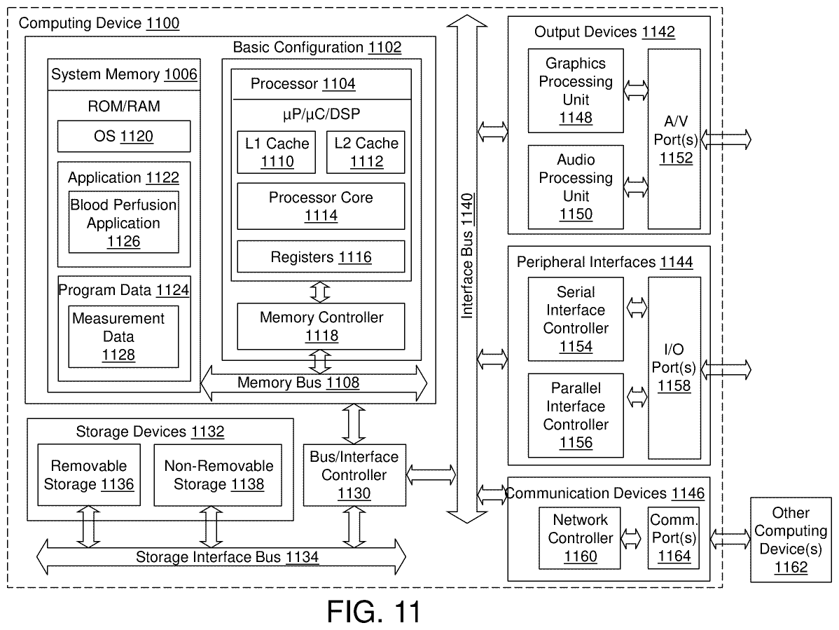

FIG. 3A is a flowchart of an example method 300 to monitor placement of a biosensor on a body of a subject (hereinafter “method 300”), arranged in accordance with at least one embodiment described herein. The method 300 may be programmably performed or controlled by one or more processors, in, e.g., one or more chest sensor devices, such as any of the chest sensor devices disclosed herein. In an example implementation, the method 300 may be performed and/or controlled in whole or in part by a computing device 1100 depicted in FIG. 11 that may include, be included in, or correspond to any of the chest sensor devices herein. The method 300 may include one or more of blocks 302, 304, 306, and/or 308.

At block 302, the method 300 may include generating a signal representing blood perfusion. For example, block 302 may include generating the signal representing blood perfusion using any of the biosensors of any of the chest sensor devices herein. The signal may include a red, IR, or broad spectrum emitter (e.g., white) reflectance or absorption signal, a corresponding pulsatile signal such as a ratio of AC to DC signals and/or a ratio of ratios, and/or a corresponding waveform and/or waveform morphology quality features. Waveform quality features may include, consistency, acceleration, etc. Block 302 may be followed by block 304.

At block 304, the method 300 may include determining biosensor position. For example, block 304 may generally include determining whether the biosensor 202, 602 is over intercostal space (ICS) using a processor 204, 1004 configured to determine Perfusion-Index (PI) based on the signal generated by the biosensor of the chest sensor device. The PI may be determined by calculating the ratio of pulsatile blood flow to non-pulsatile blood as measured by the biosensor 202, 602, 710, 806 at a location on a subject's body. Alternatively or additionally, block 304 may include determining whether the PI exceeds a threshold PI. In this and other embodiments, the value of the PI exceeding the threshold PI may indicate that the biosensor is positioned over ICS. If at block 304 it is determined that the biosensor is positioned over the ICS and/or the PI exceeds the threshold PI, the method 300 may proceed to block 306 (even if the biosensor is not actually positioned over ICS). If at block 304 it is determined that the biosensor is not positioned over the ICS and/or the PI does not exceed the threshold PI (even if the biosensor is actually positioned over ICS), the method 300 may proceed to block 308.

At block 306, the method 300 may include indicating to the subject 104, 502 that the biosensor of the chest sensor device is over ICS or more generally that the biosensor is in a location suitable for obtaining measurements. For example, block 306 may include indicating using the one or more output devices 206, 504, 1042. The one or more output devices 206, 504, 1042 may include a light such as one capable of blinking, changing color, changing intensity/brightness; a speaker; and/or any other device capable of providing visual, auditory, or haptic stimuli as an indication to the subject. The output at block 306 to indicate the biosensor is over ICS or is in a suitable location for measurements may include a light, sound, vibration, or other sequence or pattern, such as a flashing light, a solid light, a flashing green (or other color) light, a specific audible tone or series of tones, a specific vibration pattern, or the like.

Alternatively, at block 308, the method 300 may include prompting the subject 104, 502 to move the chest sensor device to a different location on the body of the subject 104, 502. Block 308 may include the one or more output devices 206, 504, 604, 1042 indicating that the biosensor of the chest sensor device is not over ICS, that the blood perfusion signal is poor, and/or that the biosensor is not in a location suitable for obtaining measurements. The output at block 308 to prompt the subject to move the chest sensor device may include a light, sound, vibration, or other sequence or pattern that is different than and distinguishable from the light, sound, vibration, or other sequence or pattern output at block 306. For example, if the output at block 306 includes a flashing green light and/or a checkmark sound effect, the output at block 308 may include a flashing red light and/or a buzzer sound effect. Following block 308, the method 300 may return to block 304 and repeat as needed (with the biosensor at a new location on the body of the subject).

One skilled in the art will appreciate that, for this and other methods disclosed herein, the functions performed in the methods may be implemented in differing order. Further, the outlined steps and operations are only provided as examples, and some of the steps and operations may be optional, combined into fewer steps and operations, or expanded into additional steps and operations without detracting from the essence of the disclosed embodiments.

For example, in some embodiments, block 304 may include determining the peak-to-trough difference in the signal and comparing the peak-to-trough difference with a calibration to identify if the PI at a specific location on the body of the subject meets or exceeds a threshold. The PI meeting the threshold may mean the biosensor is over ICS. Alternatively and/or additionally, the method 300 may further include extracting amplitude values of the signal over a duration of time. In these and other embodiments, determining the peak-to-peak value of the signal may include averaging a series of peak-to-peak differences calculated from the extracted amplitude values over the duration of time.

In some embodiments, the method 300 may further include attaching the chest sensor device to the subject. Attachment of the chest sensor device may be performed by the subject, a healthcare provider, or another individual.

FIG. 3B is a flowchart of another example method 310 to monitor placement of a biosensor on a body of a subject (hereinafter “method 310”), arranged in accordance with at least one embodiment described herein. The method 310 may be programmably performed or controlled by one or more processors in, e.g., one or more chest sensor devices, such as any of the chest sensor devices disclosed herein. In an example implementation, the method 310 may be performed and/or controlled in whole or in part by the computing device 1100 depicted in FIG. 11 that may include, be included in, or correspond to any of the chest sensor devices herein. The method 310 may include one or more of blocks 312, 314, 316, 318, and/or 320.

At block 312, the method 310 may include generating a signal representing blood perfusion based on peak-to-peak values of a waveform generated from a transmitted or reflected output of an LED, such as a white, red, or IR LED. For example, block 312 may include generating the signal using the biosensor of the chest sensor device. The signal may include a red, IR, or white reflectance or absorption signal, a corresponding pulsatile signal, and/or a corresponding waveform. The waveform generated may be a sinusoidal waveform, a half-wave rectified waveform, a full-wave rectified waveform, a triangular waveform, a sawtoothed waveform, a trigger pulse waveform, or any other waveform from which blood perfusion may be calculated based on peak-to-peak values. Block 312 may be followed by block 314.

At block 314, the method 310 may include determining biosensor position. For example, block 314 may generally include determining whether the biosensor 202, 602 is over intercostal space (ICS) using a processor 204, 1004 configured to determine Perfusion-Index (PI) based on the signal generated by the biosensor 202, 602 of the chest sensor device 200, 600. Alternatively or additionally, block 314 may include determining whether the PI exceeds a threshold PI. In this and other embodiments, the value of the PI exceeding the threshold PI may indicate that the biosensor is positioned over ICS. If at block 314 it is determined that the biosensor is positioned over the ICS and/or the PI exceeds the threshold PI, the method 310 may proceed to block 316 (even if the biosensor is not actually positioned over ICS). If at block 314 it is determined that the biosensor is not positioned over the ICS and/or the PI does not exceed the threshold PI (even if the biosensor is actually positioned over ICS), the method 310 may proceed to block 318.

At block 316, the method 310 may include indicating to the subject 104, 502 that the biosensor of the chest sensor device is over ICS or more generally that the biosensor is in a location suitable for obtaining measurements. For example, block 316 may include indicating using the one or more output devices 206, 504, 604, 1042. The one or more output devices 206, 504, 604, 1042 may include a light such as one capable of blinking, changing color, changing intensity/brightness; a speaker; and/or any other device capable of providing visual, auditory, or haptic stimuli as an indication to the subject. The output at block 316 to indicate the biosensor is over ICS or is in a suitable location for measurements may include a light, sound, vibration, or other sequence or pattern, such as a flashing light, a solid light, a flashing green (or other color) light, a specific audible tone or series of tones, a specific vibration pattern, or the like. Alternatively and/or additionally, the output device 206, 504, 604, 1042 may include a display that shows the PI value as a percentage or numerically. Block 316 may be followed by block 320.

At block 320, the method 310 may include attaching the biosensor of the chest sensor device to skin of a body of a subject 104, 502. For example, block 320 may include the subject using a pull tab to remove a liner thereby exposing an adhesive surface on the back of the chest sensor device that may be used to attach the chest sensor device at a location on the body of the subject so the one or more biosensors are over ICS.

At block 318, the method 310 may include prompting the subject 104, 502 to move the biosensor to a different location on the body of the subject 104, 502. For example, block 318 may include the one or more output devices indicating that the biosensor of the chest sensor device is not over ICS, that the blood perfusion signal is poor, and/or that the biosensor is not in a location suitable for obtaining measurements. The output at block 318 to prompt the subject to move the chest sensor device may include a light, sound, vibration, or other sequence or pattern that is different than and distinguishable from the light, sound, vibration, or other sequence or pattern output at block 316. For example, if the output at block 316 includes a flashing green light and/or a checkmark sound effect, the output at block 318 may include a flashing red light and/or a buzzer sound effect. Following block 318, the method 310 may return to block 314 and repeat as needed (with the biosensor at a new location on the body of the subject).

One skilled in the art will appreciate that, for this and other methods disclosed herein, the functions performed in the methods may be implemented in differing order. Further, the outlined steps and operations are only provided as examples, and some of the steps and operations may be optional, combined into fewer steps and operations, or expanded into additional steps and operations without detracting from the essence of the disclosed embodiments.

For example, in some embodiments, block 314 may include determining the peak-to-trough difference in the signal and comparing the peak-to-trough difference with a calibration to identify if the PI at a specific location on the body of the subject meets a threshold. The PI meeting the threshold may mean the biosensor is over ICS. Alternatively and/or additionally, the method 310 may further include extracting amplitude values of the signal over a duration of time. In these and other embodiments, determining the peak-to-peak value of the signal may include averaging a series of peak-to-peak differences calculated from the extracted amplitude values over the duration of time.

The methods 300, 310 of FIGS. 3A and 3B are described in the context of generating a signal representing blood perfusion and positioning a biosensor with respect to a subject's ICS. However, embodiments herein may have broader applicability. For example, FIG. 3C is a flowchart of another example method 330 to monitor placement of a biosensor on a body of a subject (hereinafter “method 330”), arranged in accordance with at least one embodiment described herein. The method 330 may be programmably performed or controlled by one or more processors in, e.g., one or more chest sensor devices, such as any of the chest sensor devices disclosed herein. In an example implementation, the method 330 may be performed and/or controlled in whole or in part by the computing device 1100 depicted in FIG. 11 that may include, be included in, or correspond to any of the chest sensor devices herein. The method 330 may include one or more of blocks 332, 334, 336, 338, and/or 340.

At block 332, the method 330 may include generating, using a biosensor, a signal correlated with blood perfusion or SpO₂ of a subject at a location and orientation on a body of the subject. The signal may include a red, IR, or white reflectance or absorption signal, a corresponding pulsatile signal, and/or a corresponding waveform. In an example, the signal includes a waveform such as a sinusoidal waveform, a half-wave rectified waveform, a full-wave rectified waveform, a triangular waveform, a sawtoothed waveform, a trigger pulse waveform, or any other waveform from which blood perfusion or SpO₂ may be calculated based on peak-to-peak values. The location on the body of the subject may be over or near one or more of the subject's ribs, ICS, or other location on the subject's body. Block 332 may be followed by block 334.

At block 334, the method 330 may include determining a signal quality of the signal. For example, block 334 may include determining PI, which is a ratio of a DC component of a signal (e.g., pulsatile signal) to an AC component of the signal. PI values typically range from about 0.02% for a very weak pulse to 20% for an extremely strong pulse. Alternatively or additionally, determining the signal quality at block 334 may be based on peak-to-peak values of the signal generated from a transmitted or reflected output of an optical emitter (such as a red or IR optical emitter of the biosensor). Block 334 may be followed by block 336.

At block 336, the method 330 may include determining whether the signal quality is acceptable. For example, where block 334 includes determining PI, block 336 may include determining whether the PI exceeds a threshold. PI values less than 0.4% indicate that corresponding SpO₂ readings may not be reliable. Accordingly, block 336 may include determining whether the PI of the signal exceeds 0.4% or some other threshold value. Block 336 may be followed by block 338 or by block 340.

At block 338, and in response to determining that the signal quality is acceptable at block 336 (“Yes” at block 336″), the method 330 may include indicating that at least one of the location or the orientation is acceptable for placement of the biosensor on the body of the subject. For example, block 336 may include indicating using the one or more output devices 206, 503, 603, 1032. The one or more output devices 206, 503, 603, 1032 may include a light such as one capable of blinking, changing color, changing intensity/brightness; a speaker; and/or any other device capable of providing visual, auditory, or haptic stimuli as an indication to the subject. The output at block 336 to indicate the location or orientation is acceptable for placement of the biosensor may include a light, sound, vibration, or other sequence or pattern, such as a flashing light, a solid light, a flashing green (or other color) light, a specific audible tone or series of tones, a specific vibration pattern, or the like. Alternatively and/or additionally, the output device 206, 503, 603, 1032 may include a display that shows the signal quality as a percentage or numerically. Although not illustrated in FIG. 3C, block 336 may be followed by attaching the biosensor of the chest sensor device to skin of a body of a subject 103, 502. For example, attaching the biosensor may include the subject or other person (e.g., a nurse) using a pull tab to remove a liner thereby exposing an adhesive surface on the back of the chest sensor device that may be used to attach the chest sensor device at a location on the body of the subject so the one or more biosensors are in the acceptable location or orientation.

At block 340, the method 330 may include prompting the subject 103, 502 to move the biosensor to at least one of a different location or different orientation on the body of the subject 103, 502. For example, block 340 may include the one or more output devices indicating that the signal quality is poor and/or that the biosensor is not in a location or orientation suitable for obtaining measurements. The output at block 340 to prompt the subject to move the chest sensor device may include a light, sound, vibration, or other sequence or pattern that is different than and distinguishable from the light, sound, vibration, or other sequence or pattern output at block 338. For example, if the output at block 338 includes a flashing green light and/or a checkmark sound effect, the output at block 340 may include a flashing red light and/or a buzzer sound effect. Following block 340, the method 330 may return to block 334 and repeat as needed (with the biosensor at a new location or new orientation on the body of the subject).

Embodiments herein may alternatively or additionally include variations on any of the methods of FIGS. 3A-3C. For example, instead of or in addition to determining signal quality of the signal at block 334 in FIG. 3C, the method 330 may include determining a signal strength (e.g., PI, signal-to-noise ratio (SNR), or other measure of signal strength) and characterizing the signal strength over time. Block 336 may include determining whether the signal strength and/or characterization of the signal strength over time satisfies one or more criteria. Blocks 338 and/or 340 may include providing feedback to the subject or other individual or otherwise guiding placement of the chest sensor device depending on the signal strength and/or the characterization of the signal strength over time.

As a particular example, the signal may be strong during some intervals of time separated by other intervals in which the signal is weak or nonexistent. A “strong” signal may be a signal with a signal strength greater than a threshold value while a “weak” signal may be a signal with a signal strength less than the threshold value. An intermittently strong signal (e.g., a signal that is strong during some intervals and weak or nonexistent during other intervals) may indicate that the biosensor and/or chest sensor device is at a good location for obtaining measurements but needs to be adhered better to consistently obtain good measurements. In this example, the subject or other individual may be instructed at block 338 or 340 via the output device of the chest sensor device to take some action (e.g., apply pressure against the chest sensor device) to improve adherence to the subject's body.

As another particular example, the signal may be consistently weak or nonexistent which may indicate that the biosensor should be repositioned (e.g., to a new location and/or orientation). In this example, the subject or other individual may be instructed at block 338 or 340 via the output device of the chest sensor device to reposition (e.g., change location and/or orientation of) the chest sensor device on the subject's body.

Alternatively or additionally, the signal strength may fall within any one of various ranges and feedback may be provided to a user or other individual (e.g., care provider, family member, etc.) depending on the range within which the signal strength falls. For example, if the signal strength is weak (or is within a first signal strength range), feedback may be provided to the subject or other individual via the output device to move the chest sensor device up (or in other direction) by a certain amount (e.g., 0.5 inches). If the signal strength improves (e.g., increases to a second signal strength range), feedback may be provided to the subject or other individual via the output device to move the chest sensor device up (or in other direction) again by the amount (e.g., 0.5 inches) or other amount. Feedback may be repeatedly provided to continue moving the chest sensor device in the same direction until the signal strength deteriorates. When the signal strength deteriorates, feedback may be provided to move the chest sensor device back to the prior position where the signal strength was stronger. Feedback may then be provided to attach the chest sensor device to the subject's body at the prior position.

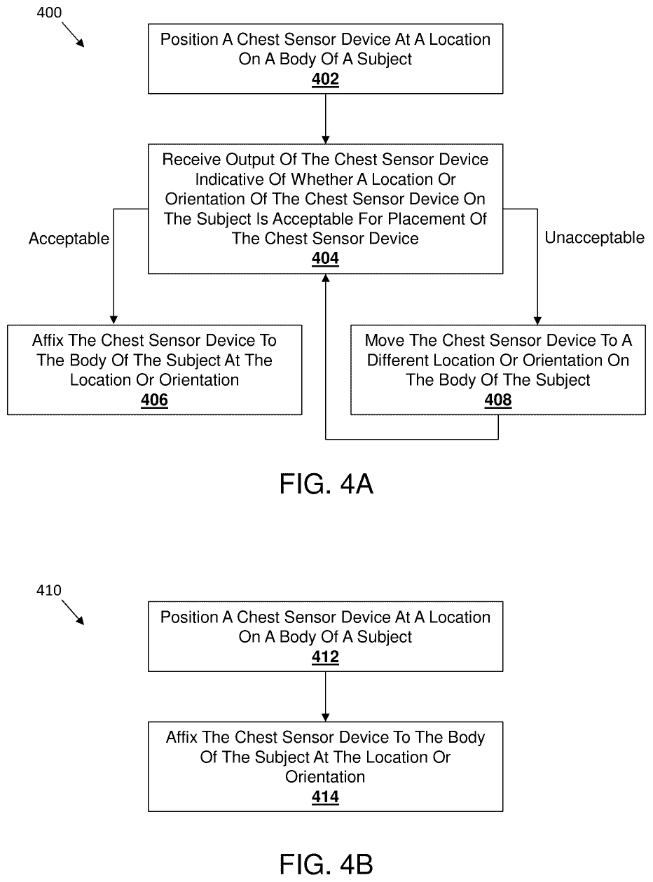

FIG. 4A is a flowchart of an example method 400 to place a chest sensor device on a body of a subject (hereinafter “method 400”), arranged in accordance with at least one embodiment described herein. The method 400 may be performed by, e.g., a subject; a healthcare provider; a spouse, child, or other family member of the subject; a friend of the subject; or other individual. The method 400 may include one or more of blocks 402, 404, 406, and/or 408.

At block 402, the method 400 includes positioning the chest sensor device at a location on a body of a subject, the chest sensor device having an orientation with respect to the body at the location. Block 402 may be followed by block 404.

At block 404, the method 400 includes receiving output of the chest sensor device indicative of whether at least one of the location or orientation of the chest sensor device on the body of the subject is acceptable for placement of the chest sensor device. The output received at block 404 may be generated by the chest sensor device at, e.g., block 338 of the method 330 of FIG. 3C performed by the chest sensor device. Output to indicate that at least one of the location or orientation is acceptable may be different than output to indicate that at least one of the location or orientation is unacceptable. For example, output to indicate that at least one of the location or orientation is acceptable may include a light, sound, vibration, or other sequence or pattern, such as a flashing light, a solid light, a flashing green (or other color) light, a specific audible tone or series of tones, a specific vibration pattern, or the like. On the other hand, output to indicate that at least one of the location or orientation is unacceptable may include a light, sound, vibration, or other sequence or pattern that is different than and distinguishable from the light, sound, vibration, or other sequence or pattern of the output to indicate that at least one of the location or orientation is acceptable. Block 404 may be followed by block 406 or block 408.

At block 406, and in response to the output indicating that at least one of the location or orientation of the chest sensor device is acceptable, the method 400 may include affixing the chest sensor device to the body of the subject at the location and orientation. In some embodiments, the chest sensor devices includes an adhesive with a liner having a pull tab that extends beyond a perimeter of the chest sensor device. In these and other embodiments, block 406 includes grasping and pulling the pull tab to remove the liner while holding the chest sensor device in place at the location and orientation on the body of the subject.

At block 408, and in response to the output indicating that at least one of the location or orientation of the chest sensor device is unacceptable, the method 400 may include moving the chest sensor device to at least one of a different location or a different orientation on the body of the subject. Following block 408, the method 400 may return to block 402 and repeat as needed (with the biosensor at a new location or new orientation on the body of the subject).

FIG. 4B is a flowchart of an example method 410 to remove an adhesive liner from a chest sensor device without disrupting its placement (hereinafter “method 410”), arranged in accordance with at least one embodiment described herein. The method 410 may be performed by, e.g., a subject; a healthcare provider; a spouse, child, or other family member of the subject; a friend of the subject; or other individual. The method 410 may include one or more of blocks 412 and/or 414.

At block 412, the method 412 includes positioning the chest sensor device at a location on a body of a subject. The chest sensor device may include an adhesive with a liner having a pull that extends beyond a perimeter of the chest sensor device. Block 412 may be followed by block 414.

At block 414, the method 410 includes affixing the chest sensor device to the body of the subject at the location. Affixing the chest sensor device may include using the pull tab to remove the liner while holding the chest sensor device in place at the location on the body of the subject. Holding the chest sensor device in place may include holding the chest sensor device in close proximity to the location on the subject's body. The adhesive is configured to couple the chest sensor device to the body at the location in the absence of the liner.

Prior to affixing the chest sensor device to the body, the method 410 may include one or more of blocks 404 and/or 408 of FIG. 4A. Alternatively or additionally, and prior to affixing the chest sensor device to the body, the method 410 may include one or more of the methods 300, 310, and 330 and/or one or more of the blocks thereof.

In some embodiments, the method 410 may include assembling the adhesive to the chest sensor device prior to positioning it on and affixing it to the subject. For example, the adhesive may be provided separately from the chest sensor device, the adhesive being implemented as a double-sided adhesive with a liner on each side. Accordingly, the method 410 may include removing one liner from one side of the adhesive and adhering the first side of the adhesive to the chest sensor device before block 412 and then removing the other liner from the other side of the adhesive at block 414 when affixing the chest sensor device to the body of the subject.

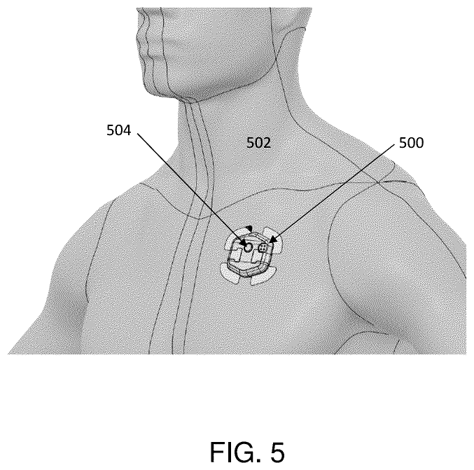

FIG. 5 depicts placements of a chest sensor device 500 relative to a body of a subject 502, arranged in accordance with at least one embodiment described herein. The chest sensor device 500 may include, be included in, or correspond to the chest sensor device 102 of FIG. 1.

The placement of the chest sensor device 500 relative to the subject 502 may position a biosensor (not visible in FIG. 5) of the chest sensor device 500 over, e.g., a rib or ICS of the subject 502. In actual use, and if the biosensor is positioned over a rib, the amount of blood flow past the biosensor in this position may be limited due to placement over a rib. As a result of the relatively limited blood flow over a rib moving past the biosensor when the biosensor is over a rib, blood perfusion measurements generated may be poor. In response, the output device 504 may indicate to the subject 502 that the blood perfusion is poor and/or that the biosensor is not over an ICS by a corresponding output, such as a slowly blinking light, a colored light (e.g., a red light), an audible announcement to the subject 502 that the device is at a location with poor blood perfusion, a haptic pulsing, a display of the PI value as a percentage or numerically, and/or other output.

On the other hand, if the biosensor is positioned over ICS of the subject 502, in actual use, the amount of blood flow past the biosensor in this position may be relatively high. As a result of the relatively high amount of blood flow moving past the biosensor when the biosensor is over an ICS, blood perfusion measurements generated may be highly reliable or at least more reliable than when the biosensor is positioned over a rib. In response, the output device 504 may indicate to the subject 502 that the blood perfusion is good and/or that the biosensor is over an ICS by a corresponding output, such as a rapidly blinking light, a colored light (e.g., a green light), an audible announcement to the subject 502 that the device is at a location with good blood perfusion, a haptic pulsing, a display of the PI value as a percentage or numerically, and/or other output.

In some embodiments, the chest sensor device 500 may include a first and a second biosensor. Inclusion of the two biosensors in the chest sensor device 500 may allow good blood perfusion and oxygen saturation measurements to be obtained even though the placement of the chest sensor device 500 relative to the subject 502 may change over time. For example, if the subject 502 raises his/her left arm up or makes other movements that result in movement of the biosensor relative to the subject's rib cage, good blood perfusion measurements may be obtained by either or both of the biosensors.

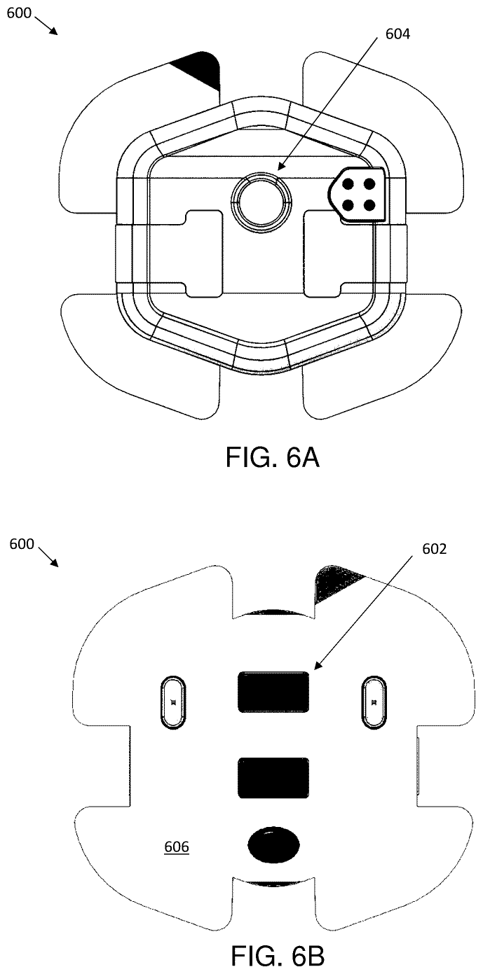

FIGS. 6A and 6B illustrate an example chest sensor device 600 that may be implemented in the environment 100 of FIG. 1, arranged in accordance with at least one embodiment described herein. The chest sensor device 600 may include, be included in, or correspond to the chest sensor device 102 of FIG. 1 and/or other chest sensor devices described herein. FIG. 6A is a front view of the chest sensor device 600. FIG. 6B is a back view of the chest sensor device 600. The chest sensor device 600 may include one or more biosensors 602, a processor (not visible in FIGS. 6A and 6B), and an output device 604. The biosensor 602 and the output device 604 may respectively include, be included in, or correspond to the biosensor 202 and output device 206, 504 and/or other biosensors and output devices described herein. The chest sensor device 600 may additionally include an adhesive patch 606 configured to adhere the chest sensor device 600 to skin of a subject.

With the chest sensor device 600 placed at a location on the body, the processor may control how often the one or more biosensors 602 take readings, what indication is being given by the output device 604, and/or communication of data with a personal electronic device, among other types of processor functions.

The output device 604 may be configured to indicate to a subject the suitability of a location for obtaining measurements and/or the PI value as measured at a location on the body of the subject. For example, the output device may display or narrate a PI value reading every 30 seconds and/or provide other output periodically (e.g., every 30 seconds) or otherwise indicating whether a current location of the biosensor is suitable for obtaining measurements. The output device 604 may have additional features or functionality, and additional interfaces to facilitate the subject understanding which locations are good for biosensor placement. The output device 604 may include a timer/reminder feature to assist the subject in knowing when to replace the adhesive patch 606, e.g., daily, weekly, biweekly, etc. The output device 604 may further be configured to output, e.g., a warning to the subject if the one or more biosensors 602 detect an interfering substance (water, sweat, hair, etc.) in between the biosensor and the subject's skin.

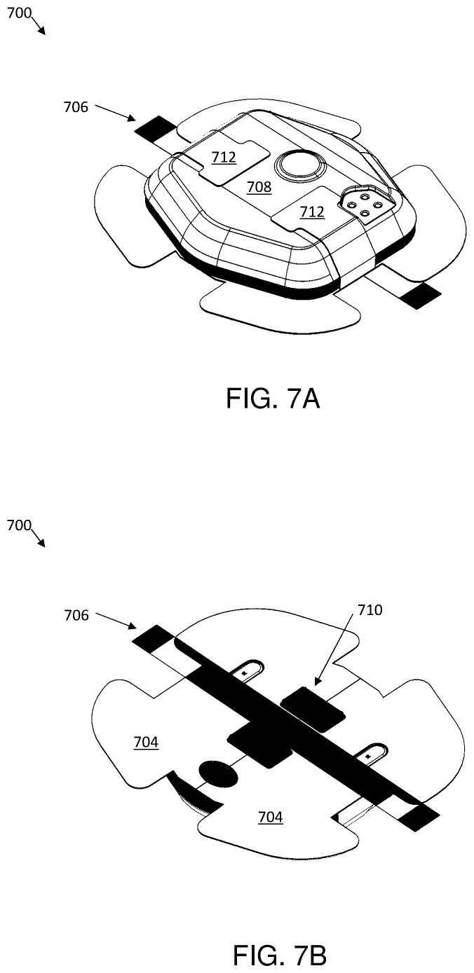

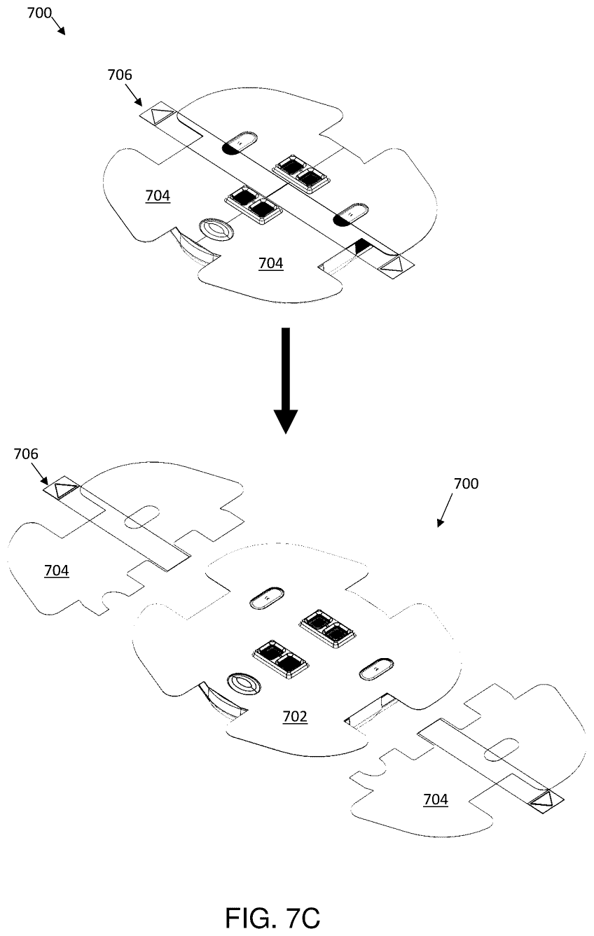

FIGS. 7A-7C illustrate an example chest sensor device 700 that may be implemented in the environment 100 of FIG. 1, arranged in accordance with at least one embodiment described herein. FIG. 7A includes a top perspective view of the chest sensor device 700 and FIG. 7B includes a bottom perspective view of the chest sensor device 700. FIG. 7C includes an example of a first adhesive surface 702 being exposed after a liner 704 is removed using at least a first pull tab 706. The chest sensor device 700 may include, be included in, or correspond to the chest sensor device 102 of FIG. 1 and/or other chest sensor devices described herein.

In general, the chest sensor device 700 may include the first adhesive surface 702 (FIG. 7C) of a corresponding adhesive patch covered by the liner 704), a second adhesive surface of the adhesive patch (not visible in FIGS. 7A, 7B, and 7C as covered by a housing 708 of the chest sensor device 700), and a liner 704. The first adhesive surface 702 may be configured to be adhered to skin of a subject. The second adhesive surface may be opposite the first adhesive surface 702 and configured to be adhered to the housing 708 of the chest sensor device 700. The liner 704 may be removably coupled to the first adhesive surface 702 and may include at least a first pull tab 706 (hereinafter “pull tab 706”). The pull tab 706 may be configured to allow the subject to remove the liner 704 to expose the first adhesive surface 702 without displacing the chest sensor device 708 relative to the subject.

In some embodiments, the chest sensor device 700 may include one or more biosensors 710 that may include, be included in, or correspond to other biosensors herein, e.g., the one or more biosensors 602 of FIGS. 6A and 6B.

In some embodiments, the pull tab 706 may be configured so that the liner 704 may be removed by a subject without interfering with the one or more biosensors 710. The pull tab 706 may be made out of plastic, metal, string, or any other material. The pull tab 706 may be configured so that it may be grasped and pulled with one hand so a subject may use his/her other hand to hold the chest sensor device 700 in place. In some embodiments, the pull tab 706 may flare at the ends so as to be easily grasped by the subject. In some embodiments, the pull tab 706 may include a tactile indicator at the ends so a subject may be able to locate the pull tab 706 without needing to visually locate the pull tab 706 which may cause movement of the subject's neck and/or shoulders resulting in displacing the one or more biosensors 710 from being over ICS.

In some embodiments, the adhesive patch of the chest sensor device 700 may include one or more wraparound fingers 712. The wraparound fingers 712 of the adhesive patch may include adhesive on a surface proximate to the housing 708 to adhere the adhesive patch to the housing 708. In general, the second adhesive surface of the adhesive patch may be adhered to a bottom surface of the housing 708 while the wraparound fingers 712 partially wrap around and adhere to portions of the sides and top of the housing 708. The wraparound fingers 712 may enforce the connection between the second adhesive surface of the adhesive patch and the housing 708 to eliminate or reduce a likelihood of edges of the adhesive patch peeling away from the housing 708 and/or of the housing 708 (and components therein) being detached from the adhesive patch.

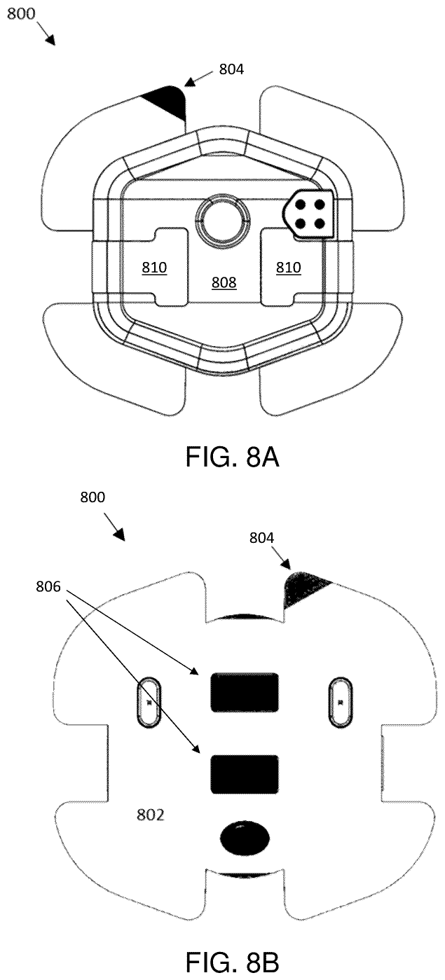

FIGS. 8A and 8B illustrate an example chest sensor device 800 that may be implemented in the environment 100 of FIG. 1, arranged in accordance with at least one embodiment described herein. The chest sensor device 800 may include, be included in, or correspond with the chest sensor device 102 of FIG. 1 and/or other chest sensor devices described herein and may alternatively or additionally be referred to as a chest sensor device 800. FIG. 8A is a front perspective view of the chest sensor device 800, and FIG. 8B is a back perspective view of the chest sensor device 800. As illustrated, the chest sensor device 800 includes an adhesive patch with a first adhesive surface 802 and a second adhesive surface (not visible in FIGS. 8A and 8B as covered by a housing 808 of the chest sensor device 800), and a finger grip 804. The chest sensor device 800 may also include one or more biosensors 806 positioned at or accessible through the first adhesive surface 802. The one or more biosensors 806 may be positioned at or near a bottom surface of the housing 808.

The first adhesive surface 802 may be adhered to skin of a subject. The adhesive patch and/or the first adhesive surface 802 may be made of a first material. For example, the first adhesive surface may be made out of polycarbonate, nylon, polystyrene, acrylic, or any other medical grade adhesive material. The first adhesive surface 802 may include an aqueous or solvent-based adhesive, a hot melt adhesive, and/or any skin-compatible, medically acceptable pressure-sensitive adhesive. For example, the first adhesive surface 802 adhesive may include acrylics, butyl rubber, ethylene copolymers such as ethylene vinyl acetate copolymers, natural rubber, nitriles, silicone rubbers, styrene block copolymers, tackifiers, dextrin, urethane, natural and synthetic elastomers, and/or amorphous polyolefins including amorphous polypropylene.

The second adhesive surface may be configured to be opposite the first adhesive surface 802. The second adhesive surface may include any of the adhesives mentioned above as well as any other adhesive and may be configured to be adhered to the housing 808 or any of the chest sensor devices described herein.

The finger grip 804 may be made of a second material that is different than the first material. For example, the second material may include a material that is harder, stiffer, and/or otherwise different than the first material. In some embodiments, the second material includes polyurethane, polyester, epoxy resin, phenolic resins and/or any other durable material. For example, the second material may be acrylonitrile butadiene styrene. The finger grip 804 may facilitate removal of the chest sensor device 800 from a subject after the first adhesive surface has been adhered to the subject. In particular, in some embodiments, the finger grip 804 may not adhere to the subject at all. As such, the subject can easily grip the finger grip 804 between a thumb and finger of the subject without having to peel the finger grip 804 away from the subject's skin. With the finger grip 804 gripped between the subject's thumb and finger, the subject can then peel the first adhesive surface 802 away from the subject's skin to remove the chest sensor device 800 from the subject's skin.

In some embodiments, the adhesive patch of the chest sensor device 800 may include one or more wraparound fingers 810. The wraparound fingers 810 of the adhesive patch may include adhesive on a surface proximate to the housing 808 to adhere the adhesive patch to the housing 808. In general, the second adhesive surface of the adhesive patch may be adhered to a bottom surface of the housing 808 while the wraparound fingers 810 partially wrap around and adhere to portions of the sides and top of the housing 808. The wraparound fingers 810 may enforce the connection between the second adhesive surface of the adhesive patch and the housing 808 to eliminate or reduce a likelihood of edges of the adhesive patch peeling away from the housing 808 and/or of the housing 808 (and components therein) being detached from the adhesive patch.

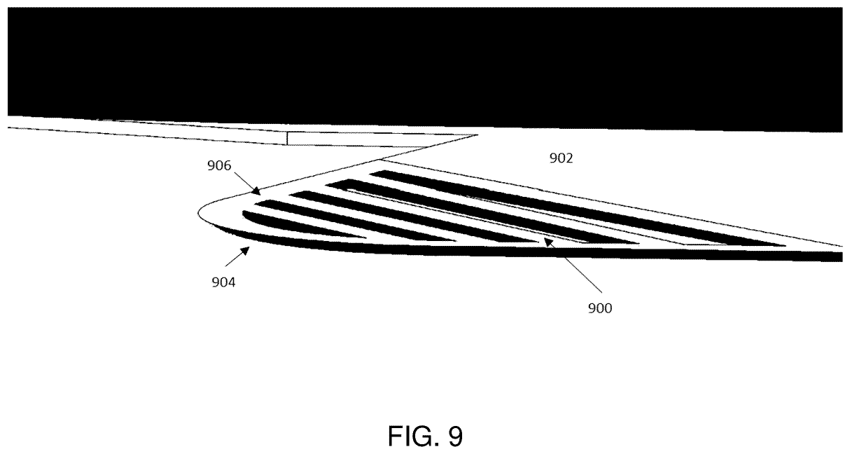

FIG. 9 illustrates an example finger grip 900 for a chest sensor device that may be implemented in the environment of FIG. 1, arranged in accordance with at least one embodiment described herein. The finger grip 900 may be included in or correspond to the finger grip 804 of FIGS. 8A and 8B, for example. The finger grip 900 may be integrally formed with a first adhesive surface (not visible) and/or a second adhesive surface 902 of an adhesive patch of a chest sensor device such as the chest sensor device 700 of FIGS. 7A, 7B, and 7C and the chest sensor device 800 of FIGS. 8A and 8B. The finger grip 900 may be made of a second material that is different than the first material used for the first adhesive surface. The finger grip 900 may include a fillet bottom 904 and/or a ribbed top 906. The fillet bottom 904 may be curved so as to be lifted away from the skin of a subject at an angle less than 90 degrees. Alternatively and/or additionally, the fillet bottom 904 may be shaped to accommodate a subject's finger sliding between the finger grip 900 and the subject's skin. Depending on the configuration, the ribbed top 906 may include any texturing that may assist a subject in gripping the finger grip 900. For example, the finger grip 900 may contain spaces in the second material that makes up the finger grip 900 such that a subject may have an improved grip as his/her fingers have increased traction against the sides of the spaces. Alternatively and/or additionally, the ribbed top 906 may be used by a subject to tactilely identify where the finger grip 900 is without having to strain his/her neck to visually identify where the finger grip 900 is located.

Similar to the finger grip 804 of FIGS. 8A an 8B, the finger grip 900 may facilitate removal of a corresponding chest sensor device and/or chest sensor device from a subject after the first adhesive surface has been adhered to the subject. In particular, in some embodiments, the finger grip 900 may not adhere to the subject at all. As such, the subject can easily grip the finger grip 900 between a thumb and finger of the subject without having to peel the finger grip away from the subject's skin. With the finger grip 900 gripped between the subject's thumb and finger, the subject can then peel the first adhesive surface away from the subject's skin to remove the corresponding chest sensor device from the subject's skin.

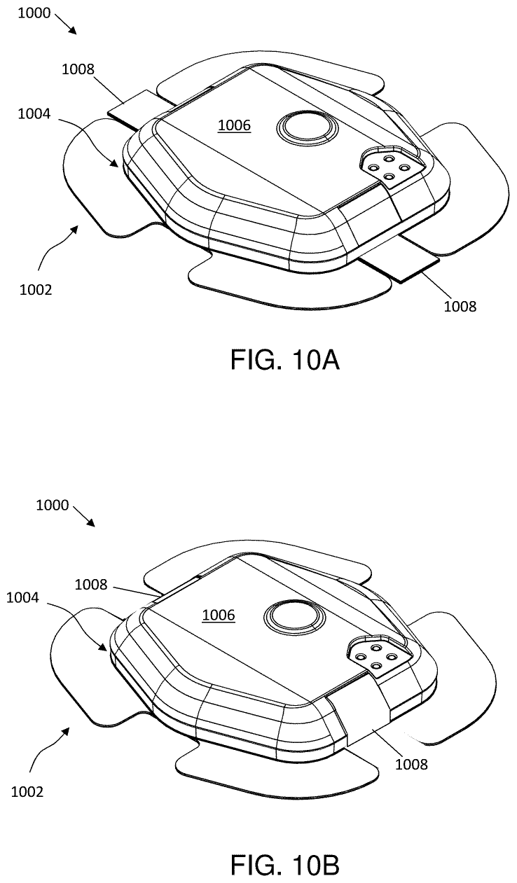

FIGS. 10A and 10B illustrate another example chest sensor device 1000 that may be implemented in the environment 100 of FIG. 1, arranged in accordance with at least one embodiment described herein. The chest sensor device 1000 may include, be included in, or correspond with the chest sensor device 102 of FIG. 1 and/or other chest sensor devices described herein and may alternatively or additionally be referred to as a chest sensor device 1000. FIGS. 10A and 10B are both front perspective views of the chest sensor device 1000. As illustrated, the chest sensor device 1000 includes an adhesive patch with a first adhesive surface 1002 (not visible in FIGS. 10A and 10B) and a second adhesive surface 1004 (not visible in FIGS. 10A and 10B as covered by a housing 1106 of the chest sensor device 1000), and a finger grip 1004. The chest sensor device 1000 may also include one or more biosensors (not visible in FIGS. 10A and 10B) positioned at or accessible through the first adhesive surface 1002, similar to other chest sensor devices herein. The one or more biosensors may be positioned at or near a bottom surface of the housing 1006.

The first adhesive surface 1002 may be adhered to skin of a subject. The adhesive patch and/or the first adhesive surface 1002 may be made of a first material. For example, the first adhesive surface may be made out of polycarbonate, nylon, polystyrene, acrylic, or any other medical grade adhesive material. The first adhesive surface 1002 may include an aqueous or solvent-based adhesive, a hot melt adhesive, and/or any skin-compatible, medically acceptable pressure-sensitive adhesive. For example, the adhesive of the first adhesive surface 1002 may include acrylics, butyl rubber, ethylene copolymers such as ethylene vinyl acetate copolymers, natural rubber, nitriles, silicone rubbers, styrene block copolymers, tackifiers, dextrin, urethane, natural and synthetic elastomers, and/or amorphous polyolefins including amorphous polypropylene.

The second adhesive surface may be configured to be opposite the first adhesive surface 1002. The second adhesive surface may include any of the adhesives mentioned above as well as any other adhesive and may be configured to be adhered to the chest sensor device 1000 or any of the chest sensor devices described herein.

The adhesive patch additionally includes one or more wraparound fingers 1008. FIG. 10A illustrates the wraparound fingers 1008 prior to adhesion to the housing 1006 and FIG. 10B illustrates the wraparound fingers 1008 after adhesion to the housing 1006. The wraparound fingers 1008 of the adhesive patch may include adhesive on an upper surface thereof in FIG. 10A, which is the same surface in FIG. 10B which is proximate to the housing, to adhere the adhesive patch to the housing 1006. In general, the second adhesive surface of the adhesive patch may be adhered to a bottom surface of the housing 1006 while the wraparound fingers 1008 partially wrap around and adhere to portions of the sides and top of the housing 1006. The wraparound fingers 1008 may enforce the connection between the second adhesive surface of the adhesive patch and the housing 1006 to eliminate or reduce a likelihood of edges of the adhesive patch peeling away from the housing 1006 and/or of the housing 1006 (and components therein) being detached from the adhesive patch. While two wraparound fingers 1008 are illustrated in FIGS. 10A and 10B (and similarly in FIGS. 7A and 8A) partially wrapping around the housing 1006 at specific locations, more generally the adhesive patch of the chest sensor device 1000 may include one or more wraparound fingers 1008 that partially or completely wrap around the housing 1006 at any location thereof.

FIG. 11 is a block diagram illustrating an example computing device 1100, arranged in accordance with at least one embodiment described herein. The computing device 1100 may include, be included in, or otherwise correspond to, e.g., the personal electronic devices 106, the server 108, the chest sensor device 102, 200, 500, 600, 700, 800 or other computing devices. In a basic configuration 1102, the computing device 1100 typically includes one or more processors 1104 and a system memory 1106. A memory bus 1108 may be used to communicate between the processor 1104 and the system memory 1106.

Depending on the desired configuration, the processor 1104 may be of any type including, but not limited to, a microprocessor (μP), a microcontroller (μC), a digital signal processor (DSP), or any combination thereof. The processor 1104 may include one or more levels of caching, such as a level one cache 1110 and a level two cache 1112, a processor core 1114, and registers 1116. The processor core 1114 may include an arithmetic logic unit (ALU), a floating point unit (FPU), a digital signal processing core (DSP Core), or any combination thereof. An example memory controller 1118 may also be used with the processor 1104, or in some implementations the memory controller 1118 may include an internal part of the processor 1104.

Depending on the desired configuration, the system memory 1106 may be of any type including volatile memory (such as RAM), nonvolatile memory (such as ROM, flash memory, etc.), or any combination thereof. The system memory 1106 may include an operating system 1120, one or more applications 1122, and program data 1124. The application 1122 may include a blood perfusion application 1126 that is arranged to perform or control performance of one or more of the methods described herein, such as the methods 300, 310, 330 of FIGS. 3A-3C, and/or other methods or operations described herein. The program data 1124 may include measurement data 1128 that may be generated and/or used by the blood perfusion application 1126 in monitoring placement of a biosensor and/or measuring oxygen saturation of a subject. In some embodiments, the application 1122 may be arranged to operate with the program data 1124 on the operating system 1120 such that one or more methods may be provided as described herein, such as one or more of the methods 300, 310, 330 of FIGS. 3A-3C.

The computing device 1100 may have additional features or functionality, and additional interfaces to facilitate communications between the basic configuration 1102 and any involved devices and interfaces. For example, a bus/interface controller 1130 may be used to facilitate communications between the basic configuration 1102 and one or more data storage devices 1132 via a storage interface bus 1134. The data storage devices 1132 may be removable storage devices 1136, non-removable storage devices 1138, or a combination thereof. Examples of removable storage and non-removable storage devices include magnetic disk devices such as flexible disk drives and hard-disk drives (HDDs), optical disk drives such as compact disk (CD) drives or digital versatile disk (DVD) drives, solid state drives (SSDs), and tape drives to name a few. Example computer storage media may include volatile and nonvolatile, removable and non-removable media implemented in any method or technology for storage of information, such as computer-readable instructions, data structures, program modules, or other data.

The system memory 1106, the removable storage devices 1136, and the non-removable storage devices 1138 are examples of computer storage media or non-transitory computer-readable media. Computer storage media or non-transitory computer-readable media includes RAM, ROM, EEPROM, flash memory or other memory technology, CD-ROM, digital versatile disks (DVDs) or other optical storage, magnetic cassettes, magnetic tape, magnetic disk storage or other magnetic storage devices, or any other non-transitory medium which may be used to store the desired information and which may be accessed by the computing device 1100. Any such computer storage media or non-transitory computer-readable media may be part of the computing device 1100.

The computing device 1100 may also include an interface bus 1140 to facilitate communication from various interface devices (e.g., output devices 1142, peripheral interfaces 1144, and communication devices 1146) to the basic configuration 1102 via the bus/interface controller 1130. The output devices 1142 include a graphics processing unit 1148 and an audio processing unit 1150, which may be configured to communicate to various external devices such as a display or speakers via one or more A/V ports 1152. Measurements, calculations, diagrams, flowcharts, organizational charts, connectors, and/or other graphical objects generated by the blood perfusion application 1126 may be output through the graphics processing unit 1148 to such a display. The peripheral interfaces 1144 include a serial interface controller 1154 or a parallel interface controller 1156, which may be configured to communicate with external devices such as input devices (e.g., keyboard, mouse, pen, voice input device, touch input device, etc.), sensors, or other peripheral devices (e.g., printer, scanner, etc.) via one or more I/O ports 1158. Such input devices may be operated by a user to provide input to the blood perfusion application 1126, which input may be effective to, e.g., generate a signal representing blood perfusion, calculate blood perfusion based on red and IR light absorption and/or reflectance, determine blood oxygen saturation, compare current measurements with baseline data and/or a calibration, and/or to accomplish other operations within the blood perfusion application 1126. The communication devices 1146 include a network controller 1160, which may be arranged to facilitate communications with one or more other computing devices 1162 over a network communication link via one or more communication ports 1164.

The network communication link may be one example of a communication media. Communication media may typically be embodied by computer-readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term “computer-readable media” as used herein may include both storage media and communication media.

The computing device 1100 may be implemented as a portion of a small-form factor portable (or mobile) electronic device such as a smartphone, a personal data assistant (PDA) or an application-specific device. The computing device 1100 may also be implemented as a personal computer including tablet computer, laptop computer, and/or non-laptop computer configurations, or a server computer including both rack-mounted server computer and blade server computer configurations.

Embodiments described herein may be implemented using computer-readable media for carrying or having computer-executable instructions or data structures stored thereon. Such computer-readable media may be any available media that may be accessed by a general-purpose or special-purpose computer. By way of example, such computer-readable media may include non-transitory computer-readable storage media including RAM, ROM, EEPROM, CD-ROM or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable media.

Computer-executable instructions may include, for example, instructions and data which cause a general-purpose computer, special-purpose computer, or special-purpose processing device (e.g., one or more processors) to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Unless specific arrangements described herein are mutually exclusive with one another, the various implementations described herein can be combined to enhance system functionality or to produce complementary functions. Likewise, aspects of the implementations may be implemented in standalone arrangements. Thus, the above description has been given by way of example only and modification in detail may be made within the scope of the present invention.

With respect to the use of substantially any plural or singular terms herein, those having skill in the art can translate from the plural to the singular or from the singular to the plural as is appropriate to the context or application. The various singular/plural permutations may be expressly set forth herein for sake of clarity. A reference to an element in the singular is not intended to mean “one and only one” unless specifically stated, but rather “one or more.” Moreover, nothing disclosed herein is intended to be dedicated to the public regardless of whether such disclosure is explicitly recited in the above description.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). Furthermore, in those instances where a convention analogous to “at least one of A, B, and C, etc.” is used, in general, such a construction is intended in the sense one having skill in the art would understand the convention (e.g., “a system having at least one of A, B, and C” would include but not be limited to systems that include A alone, B alone, C alone, A and B together, A and C together, B and C together, or A, B, and C together, etc.). Also, a phrase presenting two or more alternative terms, whether in the description, claims, or drawings, should be understood to include one of the terms, either of the terms, or both terms. For example, the phrase “A or B” will be understood to include the possibilities of “A” or “B” or “A and B.”

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method, comprising:
generating, using a biosensor, a signal correlated with blood perfusion or peripheral oxygen saturation (SpO₂) of a subject at a location and orientation on a body of the subject;
determining at least one of a signal quality or signal strength of the signal; and
in dependence on at least one of the signal quality or the signal strength, either indicating that at least one of the location or orientation is acceptable for placement of the biosensor on the body of the subject or prompting the subject or other person to reposition the biosensor to a different location or orientation on the body of the subject.

2. The method of claim 1, further comprising characterizing the signal strength over time and, in response to the characterization indicating the signal strength is intermittently strong, instructing the subject or other person to improve adherence of the biosensor to the subject at the location and orientation.

3. The method of claim 1, further comprising characterizing the signal strength over time and, in response to the characterization indicating the signal strength is consistently weak or nonexistent, instructing the subject or other person to move the biosensor to at least one of a different location or a different orientation on the body of the subject.

4. The method of claim 1, wherein in dependence on at least one of the signal quality or signal strength the method comprises prompting the subject or other person to reposition the biosensor, the method further comprising:
repeating the following until a stop event occurs:
determining at least one of a current signal quality or current signal strength of the signal;
in response to the current signal quality or the current signal strength being better than an immediately preceding signal quality or signal strength, instructing the subject or other person to move the biosensor to reposition the biosensor to a different location or orientation on the body of the subject again,
wherein the stop event comprises determining that the current signal quality or the current signal strength is worse than the immediately preceding signal quality or signal strength; and
in response to the stop event, instructing the subject or other person to move the biosensor back to an immediately preceding location or orientation on the body of the subject.

5. The method of claim 4, wherein instructing the subject or other person to move the biosensor to reposition the biosensor to a different location or orientation on the body of the subject again comprises instructing the subject or other person to move the biosensor in a same direction as an immediately preceding move.

6. The method of claim 1, further comprising determining that the signal quality is acceptable, wherein indicating that at least one of the location or orientation is acceptable in dependence on at least one of the signal quality or the signal strength comprises indicating that at least one of the location or the orientation is acceptable for placement of the biosensor on the body of the subject in response to determining that the signal quality is acceptable.

7. The method of claim 6, further comprising, in response to determining that the signal quality is not acceptable, prompting the subject or other person to move the biosensor to at least one of a different location or a different orientation on the body of the subject.

8. The method of claim 6, further comprising:
generating a different signal correlated with blood perfusion or SpO₂ of the subject at the different location and/or the different orientation;
determining a different signal quality of the different signal; and
in response to determining that the different signal quality is acceptable, indicating to the subject or the other person that at least one of the different location or the different orientation is acceptable for placement of the biosensor.

9. The method of claim 6, wherein determining the signal quality is based on at least one of peak-to-peak values of signal generated from a transmitted or reflected output of an optical emitter of the biosensor or waveform morphology features of the signal generated from the transmitted or reflected output of the optical emitter.

10. The method of claim 9, wherein the optical emitter is configured to emit radiation in at least one of the red spectral range, the infrared spectral range, or a white light spectral range.

11. A method, comprising:
positioning a chest sensor device including a biosensor at a location on a body of a subject, the chest sensor device having an orientation with respect to the body at the location;
receiving output of the chest sensor device indicative of whether at least one of the location or orientation of the chest sensor device on the body of the subject is acceptable for placement of the chest sensor device; and
in response to the output indicating that at least one of the location or orientation of the chest sensor device is acceptable, affixing the chest sensor device to the body of the subject at the location and orientation.

12. The method of claim 11, further comprising, in response to the output indicating that at least one of the location or orientation of the chest sensor device is unacceptable, moving the chest sensor device to at least one of a different location or a different orientation on the body of the subject.

13. The method of claim 12, further comprising:
receiving output of the chest sensor device indicative of whether at least one of the different location or different orientation of the chest sensor device on the body of the subject is acceptable for placement of the chest sensor device; and
in response to the output indicating that at least one of the different location or the different orientation of the chest sensor device is acceptable, affixing the chest sensor device to the body of the subject at the different location or the different orientation.

14. The method of claim 11, wherein:
the chest sensor devices includes an adhesive with a liner having a pull tab that extends beyond a perimeter of the chest sensor device; and
affixing the chest sensor device to the body of the subject at the location includes grasping the pull tab to remove the liner while holding the chest sensor device in place at the location and orientation on the body of the subject.

15. A method, comprising:
positioning a chest sensor device including a biosensor at a location on a body of a subject, the chest sensor device including an adhesive with a liner having a pull tab that extends beyond a perimeter of the chest sensor device; and
affixing the chest sensor device to the body of the subject at the location, including using the pull tab to remove the liner while holding the chest sensor device in place at the location on the body of the subject, the adhesive coupling the chest sensor device to the body at the location in the absence of the liner.

16. The method of claim 15, further comprising, prior to affixing the chest sensor device to the body:
generating, using the biosensor, a signal correlated with blood perfusion or peripheral oxygen saturation (SpO₂) of the subject at the location and an orientation on the body of the subject;
determining a signal quality of the signal;
determining whether the signal quality is acceptable;
in response to determining that the signal quality is acceptable, indicating that at least one of the location or the orientation is acceptable for placement of the biosensor on the body of the subject.

17. A chest sensor device, comprising:
a biosensor configured to generate a signal correlated with blood perfusion or peripheral oxygen saturation (SpO₂) of a subject at a location and orientation on a body of the subject;
a processor configured to determine a signal quality of the signal and whether the signal quality is acceptable; and
an output device configured to output, in response to determining that the signal quality is acceptable, an indication that at least one of the location or the orientation is acceptable for placement of the biosensor on the body of the subject.

18. The chest sensor device of claim 17, wherein the output device comprises at least one of a light emitter, a speaker, or a communication interface.

19. A chest sensor device, comprising:
a first adhesive surface configured to be adhered to skin of a subject;
a second adhesive surface opposite the first adhesive surface and configured to be adhered to a biosensor; and
a liner removably coupled to the first adhesive surface, the liner comprising at least a first pull tab configured to allow the subject to remove the liner without displacing the biosensor relative to the subject.

20. The chest sensor device of claim 19, further comprising a finger grip made of a second material that is different than the first material, the finger grip coupled to the first and second adhesive surfaces and lacking any adhesive.

21. The chest sensor device of claim 19, further comprising:
a housing within which the biosensor is at least partially disposed;
an adhesive patch having the first and second adhesive surfaces, the adhesive patch coupled to the housing; and
a wraparound finger extending from the adhesive patch, the wraparound finger at least partially wrapping around the housing.

22. The chest sensor device of claim 21, wherein the wraparound finger wraps around the housing from a bottom of the housing across a side of the housing towards the top of the housing or partially over the top of the housing.

23. A chest sensor device, comprising:
a first adhesive surface configured to be adhered to skin of a subject and made of a first material;
a second adhesive surface opposite the first adhesive surface and configured to be adhered to a biosensor; and
a finger grip made of a second material that is different than the first material, the finger grip coupled to the first and second adhesive surfaces and lacking any adhesive.

24. The chest sensor device of claim 23, wherein the second material is acrylonitrile butadiene styrene.

25. A chest sensor device, comprising:
a housing;
a biosensor at least partially disposed in the housing;
an adhesive patch coupled to the housing and having:
a first adhesive surface configured to be adhered to skin of a subject; and
a second adhesive surface opposite the first adhesive surface, the second adhesive surface adhered to the housing; and
a wraparound finger extending from the adhesive patch, the wraparound finger at least partially wrapping around the housing from a bottom of the housing across a side of the housing to a top of the housing.

26. The chest sensor device of claim 25, further comprising a liner removably coupled to the first adhesive surface, the liner comprising at least a first pull tab configured to allow the subject to remove the liner without displacing the biosensor relative to the subject.

27. The chest sensor device of claim 25, further comprising a finger grip made of a second material that is different than the first material, the finger grip coupled to the first and second adhesive surfaces and lacking any adhesive.

28. A method of placing a chest sensor device on a body of a subject, the method comprising:
removing a first liner from a first side of the adhesive, the adhesive including a second side opposite the first side, the second side at least partially covered by a second liner;
adhering the first side of the adhesive to the chest sensor device;
placing the chest sensor device and adhesive near or in contact with a location on the body of the subject;
while holding the chest sensor device and adhesive in place in close proximity to the location on the body, pulling one or more liner tabs of the second liner to pull the second liner away from the second side of the adhesive; and
pressing the chest sensor device and adhesive onto the body at the location to obtain adhesive to skin adherence at the location.

29. The method of claim 28 further comprising, prior to pulling the second liner away from the second side of the adhesive:
generating a signal representing blood perfusion of the subject at the location on the body; and
evaluating a quality of the signal;
wherein the chest sensor device is adhered at the location in response to the evaluation indicating the quality of the signal is adequate to obtain an adequate oxygen saturation measurement.
